(19) United States
(12) Patent Application Publication
(10) Pub. No.: US 2025/0355070 A1
(43) Pub. Date: Nov. 20, 2025

(54) Correction of Interference Effects in Echo-Planar Imaging

(71) Applicant: Siemens Healthineers AG, Forchheim (DE)

(72) Inventors: Thorsten Speckner, Erlangen (DE); Manuela Rösler, Erlangen (DE)

(73) Assignee: Siemens Healthineers AG, Forchheim (DE)

(21) Appl. No.: 19/208,721

(22) Filed: May 15, 2025

(30) Foreign Application Priority Data
May 16, 2024 (DE) 10 2024 204 547.0

(51) Int. Cl. G01R 33/561; G01R 33/56; G01R 33/565

(52) U.S. Cl. CPC G01R 33/5616; G01R 33/5608; G01R 33/56518; G01R 33/56572

(57) ABSTRACT

A method for performing magnetic resonance imaging of an examination region using an echo-planar sequence, including: generating a theoretical model which represents an oscillating structure of a magnetic field generation unit, wherein as a result of an oscillation of the oscillating structure, eddy currents are induced in the oscillating structure and produce a gradient-type interference magnetic field in the examination region; determining the gradient-type interference magnetic field based on the theoretical model; applying the echo-planar sequence; receiving magnetic resonance signals from the examination region, wherein magnetic resonance raw data is generated based on the magnetic resonance signals; determining corrected magnetic resonance raw data based on the magnetic resonance raw data and based on the determined gradient-type interference magnetic field in the examination region; and reconstructing magnetic resonance image data based on the corrected magnetic resonance raw data.

### TECHNICAL FIELD

The disclosure relates to a method for performing magnetic resonance imaging of an examination region using an echo-planar sequence. The disclosure also relates to a magnetic resonance imaging system by means of which such a method can be performed.

### BACKGROUND

A magnetic resonance imaging system comprises a control device and a scanning unit. The scanning unit has a so-called magnetic field generation unit. The magnetic field generation unit comprises a basic field magnet, a gradient coil for generating phase-encoding gradients, readout gradients, and slice selection gradients, so-called shim coils for improving the homogeneity of the basic magnetic field, and a radio-frequency magnetic coil, also referred to as a body coil. A body coil can be used to transmit and/or receive radio-frequency signals.

When using echo-planar sequences (abbreviated to “EPI” sequences), the readout gradients must be switched to high gradient strengths and then off again very rapidly in order to allow rapid scanning of the lines in the k-space. In this case, interactions occur between the basic field magnet and the gradient coil, resulting in oscillations that can adversely affect the entire measurement. Furthermore, gradient-induced eddy currents are produced as a result of the rapid gradient changes, said eddy currents occurring on electrically conductive structures and, in particular, also on resonantly excited electrically conductive structures of the magnetic field generation unit, and counteracting the build-up of the gradient field. A distinction is made between primary eddy currents, which are produced as a result of the magnetic field gradients, and secondary eddy currents. Lorenz forces occur as a result of the induced primary eddy currents in the basic magnetic field. If the Lorenz forces excite mechanical resonances, secondary eddy currents are generated as a result of these mechanical oscillations. The present patent application is concerned with the compensation of the secondary eddy currents. This reduction of the gradient field has the effect that the signal maximum is not registered in the middle of the k-space, but is instead registered behind the middle of the k-space with odd phase-encoding steps and ahead of the middle of the k-space with even phase-encoding steps.

This zigzag line of the signal maxima results in the occurrence of artifacts, so-called N/2 ghosts, referred to in this particular case as “EPI ghosts” or “ghost artifacts”. These ghost artifacts superimpose the image data with a shift of N/2 in a phase-encoding direction. N/2 corresponds in this case to half of the number of phase-encoding lines that are required for the scanning of the examination region (also referred to as the “region of interest” and abbreviated to “ROI”) or of the field of view (also abbreviated to “FOV”) in a phase-encoding direction. Even if the “EPI ghosts” appear similar to wrap-around artifacts which come from magnetic resonance signals from tissues outside the FOV, said signals being generated with a frequency above the Nyquist frequency, they differ from these in that the “EPI ghosts” also occur if the examined object is smaller in a phase-encoding direction than the FOV and therefore no frequencies above the Nyquist frequency can occur.

Similar vibration-induced phenomena also occur as a result of excitation in the mechanical resonance of the gradient coil. The eddy currents can therefore be produced not only directly by rapid magnetic field changes, but also by moved electrically conductive components that are penetrated by magnetic fields. Previously applied compensation methods are not particularly effective or have other negative effects on imaging sequences that are used. For example, they increase the total acquisition time and the minimum possible echo time, as well as the susceptibility to magnetic field drift. A magnetic field drift can be caused by the warming of individual components, in particular the gradient coil or the so-called shim irons.

As a countermeasure, certain echo times are conventionally prohibited in connection with EPI sequences, in particular, echo times that correspond to natural resonances of gradient coils. However, owing to the considerable attenuation of the oscillation in the magnetic field, such an approach means that a very wide range of echo times must be prohibited. Optimization of imaging sequences is severely limited thereby.

As a further countermeasure, reference recordings for phase correction are made in the steady and/or identical mechanical state of the gradient system. The three variants of phase correction described below are known from the prior art.

In the case of internal phase correction, also referred to as “3-line B₀” correction, three reference lines are captured before each readout phase. Internal phase correction is illustrated in FIG. 1. The time for the readout of the reference lines is relatively short, but must be added to the time for the actual EPI sequence. The readout sequence for the readout of the three reference lines differs from the readout train of the actual EPI sequence for image acquisition. The additional time requirement of approximately 5 ms (ms=milliseconds) for each EPI sequence is comparatively short. Overall, internal phase correction is characterized by short measuring times and a high degree of resilience against patient movements.

In the case of external phase correction, illustrated in FIG. 2, the readout of the three reference lines involves applying the entire readout gradient, which is subsequently used for the image acquisition of the EPI sequence. Using this approach, artifact suppression is particularly strong, but the time overhead resulting from the application of the entire readout gradient is particularly high for the application and readout of the reference lines. The additional time requirement is approximately the repetition time of the EPI sequence. If there are field variations between different repetitions, the correction can be inaccurate. External phase correction is also sensitive to patient movements. Otherwise, the image quality achieved by external phase correction is increased in comparison with internal phase correction.

In the case of the third known method, illustrated in FIG. 3, the reduction of ghost artifacts is based on a GRAPPA reference recording. For this purpose, two additional reference recordings are made at the beginning of an EPI sequence, in order to record GRAPPA-like kernels with which to correct imperfections in the k-space trajectory that is scanned during the raw data acquisition. Here, likewise, the complete readout gradient is applied during the reference recording. The additional time requirement here is twice the repetition time of the actual EPI sequence. If there are field variations between different repetitions, the correction can become inaccurate.

### SUMMARY

The object is therefore to develop a method for performing magnetic resonance imaging using an echo-planar sequence, with higher image quality and greater resilience against movements, and with a lower time overhead in comparison with conventional approaches.

This object is achieved by a method for performing magnetic resonance imaging of an examination region using an echo-planar sequence and a magnetic resonance imaging system.

According to the disclosed method for performing magnetic resonance imaging of an examination region using an echo-planar sequence, a theoretical model is generated on the basis of the echo-planar sequence. In the case of an echo-planar sequence, a frequency gradient or readout gradient produces a so-called echo train with a plurality of switches, for example, 128 switches. This plurality of switches allows the data acquisition of an image in a very short time, for example, 70 ms. The echo-planar sequence is particularly suitable for the recording of dynamic physiological processes in the body of a patient. The theoretical model represents an oscillating structure of a magnetic field generation unit, where oscillation of the oscillating structure induces eddy currents in the oscillating structure, which generate a gradient-type interference magnetic field in the examination region. A gradient-type interference magnetic field is determined on the basis of the theoretical model. The echo-planar sequence is then applied. As explained in the introduction, the disclosed method compensates for the secondary eddy currents that are produced as a result of the mechanical oscillations that are produced by primary eddy currents. The primary eddy currents also mentioned are preferably compensated by a precorrection of the gradient fields that are applied.

In response to the echo-planar sequence that is applied, magnetic resonance signals from the examination region are received and captured, magnetic resonance raw data being generated on the basis of the magnetic resonance signals. Corrected magnetic resonance raw data is then determined on the basis of the gradient-type interference magnetic field that has been determined in the examination region and on the basis of the magnetic resonance raw data. By virtue of the knowledge relating to the interference magnetic field, it is possible, in particular, to make corrections to the phase response or the phase of the magnetic resonance raw data, said corrections resulting in a reduction of the N/2 ghosts described in the introduction.

Finally, magnetic resonance image data is reconstructed on the basis of the corrected magnetic resonance raw data.

In other words, according to the disclosed method, use is initially made of a theoretical model based on knowledge of the properties of the echo-planar sequence and, if applicable, system parameters of the magnetic resonance imaging system that is used for the magnetic resonance imaging. The theoretical model describes oscillations that are produced by primary eddy currents. The oscillations in turn cause Lorenz forces in the magnetic field that is present, thereby producing secondary eddy currents. An interference magnetic field is produced on the basis of these secondary eddy currents, and is determined on the basis of the theoretical model.

Departing from convention, the disclosed method involves image correction on the basis of a theoretical model by means of which the interference magnetic field is determined. In this case, known system parameters of the magnetic resonance imaging system, or known system parameter values assigned to these system parameters, are used for the calculation of the theoretical model. If applicable, these system parameters can be determined experimentally in advance. Unlike the conventional approaches, however, these system parameter values need only be experimentally determined once, if at all, and can then be used as required as model parameter values in order to calculate the interference magnetic field.

The disclosed magnetic resonance imaging system has a modeling unit for generating a theoretical model, which represents an oscillating structure of a magnetic field generation unit, on the basis of an echo-planar sequence that is to be applied. As a result of oscillations of the oscillating structure, eddy currents that produce a gradient-type interference magnetic field in an examination region are induced in the oscillating structure. The modeling unit is also used to determine the gradient-type interference magnetic field on the basis of the theoretical model.

A further part of the disclosed magnetic resonance imaging system is a sequence unit for applying the echo-planar sequence.

The disclosed magnetic resonance imaging system further comprises an input interface for receiving magnetic resonance signals from the examination region, magnetic resonance raw data being generated on the basis of said magnetic resonance signals.

The disclosed magnetic resonance imaging system comprises a correction unit for determining corrected magnetic resonance raw data on the basis of the magnetic resonance raw data and on the basis of the determined gradient-type interference magnetic field in the examination region.

The disclosed magnetic resonance imaging system additionally comprises a reconstruction unit for reconstructing magnetic resonance image data on the basis of the corrected magnetic resonance raw data. The disclosed magnetic resonance imaging system shares the advantages of the disclosed method for performing magnetic resonance imaging of an examination region using an echo-planar sequence.

A major mart of the previously cited components of the disclosed magnetic resonance imaging system can be realized wholly or partly in the form of software modules in a processor of a corresponding computing system, for example by a control device of a magnetic resonance imaging system or a computer that is used to control such a system. A largely software-based implementation has the advantage that computing systems already in use can also be upgraded easily by means of a software update in order to operate in the disclosed manner.

In this respect, the object is also achieved by a corresponding computer program product comprising a computer program which can be loaded directly into a computing system and has program sections for executing the steps of the disclosed method for performing magnetic resonance imaging of an examination region using an echo-planar sequence when the program is executed in the computing system. In addition to the computer program, such a computer program product can optionally comprise additional elements such as, for example, documentation and/or additional components, including hardware components such as, for example, hardware keys (dongles, etc.) for using the software.

For the purpose of transportation to the computing system or control device and/or storage on or in the computing system or control device, it is possible to use a computer-readable medium, for example a memory stick, a hard disk or other transportable or permanently installed data medium, on which are stored the program sections of the computer program which can be read in and executed by a computing system. The computing system can have, for example, one or more interacting processors or similar for this purpose.

The dependent claims and the following description each contain particularly advantageous aspects and developments of the disclosure. In this case, the claims in one statutory class of claims in particular can also be developed in a similar manner to the dependent claims in another statutory class of claims. Furthermore, in the context of the disclosure, the various features of different exemplary aspects and claims can also be combined to form novel exemplary aspects.

In a preferred aspect of the disclosed method for performing magnetic resonance imaging of an examination region using an echo-planar sequence, the theoretical model comprises a mechanical model that describes the mechanical oscillatory characteristics of a magnetic field generation unit by means of which the magnetic resonance imaging is performed. The oscillations of the magnetic field generation unit that are caused by the primary eddy currents during the imaging are advantageously taken into account when determining the secondary eddy currents and the interference magnetic field produced thereby.

In a preferred variant of the disclosed method for performing magnetic resonance imaging of an examination region using an echo-planar sequence, the determination of the corrected magnetic resonance raw data comprises a phase correction. A deviation of a magnetic field causes the phase of a scanned magnetic resonance signal to change. In order to correct this error, the phase of the scanned magnetic resonance signals is corrected on the basis of the theoretical model.

Further to this, the determination of the corrected magnetic resonance raw data preferably comprises a correction of the basic magnetic field B₀. The change of the basic magnetic field causes the Lamor frequency of the excited protons to change and therefore also the frequency with which magnetic resonance signals are captured. As a result of correcting the B₀ field, a shift of the image data in the image data space or position space is compensated. Since the pixel bandwidth in the readout direction is different in comparison with the pixel bandwidth in a phase-encoding direction in the case of an EPI sequence, distortion of the image data also occurs in the case of an EPI sequence, and is likewise compensated by the correction of the basic magnetic field B₀.

In an aspect of the disclosed method for performing magnetic resonance imaging of an examination region using an echo-planar sequence, the theoretical model is designed in such a way that the eddy currents comprise fast-decaying eddy currents having a time constant between 0.1 and 10 ms. This depends on the material and the structural nature of the oscillating structure. Typical values for decay times of eddy currents in oscillating components of a magnetic resonance imaging system lie in the cited value range. Time constants shorter than 0.1 ms can be disregarded since the phase error is too small in this case. Time constants longer than 10 ms would shift all k-space lines and therefore would not cause an N/2 ghost.

The theoretical model of the oscillating structure preferably comprises an attenuated harmonic oscillator. The model of the attenuated harmonic oscillator can be fully described using a small number of parameters, in particular the natural frequency and the attenuation. More complex oscillating structures also behave in approximately the same way as a harmonic oscillator in response to small displacements. The oscillatory characteristics of a magnetic resonance imaging system can advantageously be described with ease in the form of a model. Moreover, an extensive range of complete analytical solutions that describe the mechanical oscillation is available.

The echo-planar sequence preferably comprises a reference measurement for the phase change. This reference measurement preferably comprises the internal phase correction mentioned in the introduction, also referred to as “3-line B₀” correction. In this variant, the theoretical model is used to perform a correction of this reference-based internal phase correction in order to further reduce artifacts without increasing the recording time.

The reference measurement for the phase change, also referred to as a “PC scan”, preferably comprises a complete and preferably one-off reference recording of the k-space. A one-off measurement of the space makes it possible to determine k-space line-dependent correction values of the basic magnetic field and of the linear phase response or the phase. Since a clinical EPI protocol comprises a multiplicity of EPI measurements, the overall measuring time is hardly lengthened by a single additional complete measurement of the k-space without phase encoding.

The reference measurement, preferably, takes place in the steady state. For example, if a complete PC scan takes place, the internal phase correction, also referred to as “3-line B₀” correction, can take place in the steady state because settling of the oscillating structure already takes place during the first half of the PC scan. For example, if 64 k-space lines are recorded for a PC scan, the vibration during the recording of the three middle k-space lines as a reference measurement for an external phase correction is already in the steady state, but the PC scan takes place partly in the non-steady state. If the actual image recording is then performed in the steady state, the internal phase correction can be used as a correct reference measurement because it was likewise performed in the steady state. If the PC scan is started in the non-steady state, a time saving is produced.

Likewise, the echo-planar sequence particularly preferably comprises an initial reference measurement for identifying drift effects. In order to determine so-called drift effects, it can be advantageous before the imaging to perform an additional reference measurement by applying reference gradients and using the results thereof to correct system parameter values or to correct the response characteristics of the magnetic resonance imaging system determined in the form of a model. In this advantageous variant, the relatively modest time requirement for the reference measurement is combined with a markedly improved image quality as a result of the interference field calculated in the form of a model. So-called drift effects, which can result in errors during temporally extended measurements in particular, are advantageously compensated.

In an aspect of the disclosed method for performing magnetic resonance imaging of an examination region using an echo-planar sequence, a reference recording, which preferably comprises a “3-line B0” correction or an internal phase correction, is performed first. This recording allows the determination preferably of a deviation of the basic magnetic field and preferably a deviation of the phase response or a deviation of the phase, and the determination preferably also of a temporal echo-to-echo interval. This temporal echo-to-echo interval specifies the temporal interval between two consecutive echoes that are excited by a radio-frequency signal of an echo-planar sequence. This temporal echo-to-echo interval can vary slightly due to the described interference effects.

On the basis of the temporal echo-to-echo interval, a mechanical model of a magnetic field generation unit of a magnetic resonance imaging system that performs the imaging is then generated as a theoretical model. The mechanical model represents the mechanical oscillatory characteristics of the magnetic field generation unit in the form of a harmonic oscillator. On the basis of this model, it is possible to estimate the contribution of the eddy currents resulting from the oscillations to the production of the basic magnetic field in the form of a model, and it is possible to calculate the phase response or the phase of the magnetic resonance signal as a temporal shift in the time domain as a function of a k-space line and the excitation frequency.

On the basis of this model, an extended correction is performed in respect of the previously estimated deviation of the basic magnetic field and the previously estimated deviation of the phase response of the magnetic resonance signal, and corrected values for the basic magnetic field and the phase response or the phase are calculated thus as a function of the i^{-th} k-space line.

Lastly, a phase correction of the magnetic resonance raw data and, therefore, also of the image data takes place on the basis of the corrected field data and phase data.

In a particularly preferred variant of the disclosed method for performing magnetic resonance imaging of an examination region using an echo-planar sequence, additionally and before determining the theoretical model, one-off use is made of a complete phase change scan or a phase scan recorded in the steady state, and the k-space line-dependent correction values are determined on the basis of the measured data produced thus. The measured data is obtained from the examination region. The values thus produced are used as basic values for the theoretical model of the magnetic field generation unit.

In a particularly preferred variant of the disclosed method for performing magnetic resonance imaging of an examination region using an echo-planar sequence, the echo-planar sequence comprises a reference measurement for the phase change, a temporal echo-to-echo interval is determined on the basis of the reference measurement, and the theoretical model is determined on the basis of said temporal echo-to-echo interval. Advantageously, the

In a preferred aspect of the disclosed method for performing magnetic resonance imaging of an examination region using an echo-planar sequence, the echo-planar sequence has a reference measurement for the phase change, which reference measurement includes a scan of at least three reference lines. The reference measurement preferably has precisely three reference lines. The recording of only three reference lines in readout direction makes it possible to reduce the time of the reference measurement in comparison with a reference measurement that covers the entire k-space of the imaging. Using three reference lines in the readout direction, it is possible at least to determine approximate information with respect to a phase change between the individual reference lines.

### BRIEF DESCRIPTION OF THE DRAWINGS

The disclosure is explained again in greater detail below with reference to exemplary aspects and the appended figures, in which:

FIG. 1 shows a schematic illustration of a pulse sequence diagram of an internal phase correction,

FIG. 2 shows a schematic illustration of a pulse sequence diagram of an external phase correction,

FIG. 3 shows a schematic illustration of a pulse sequence diagram of a phase correction which takes place on the basis of a GRAPPA reference recording,

FIG. 4 shows a flow diagram which illustrates a method for performing magnetic resonance imaging using an echo-planar sequence in accordance with an exemplary aspect of the disclosure,

FIG. 5 shows a flow diagram which illustrates a method for performing magnetic resonance imaging using an echo-planar sequence in accordance with an alternative exemplary aspect of the disclosure,

FIG. 6 shows a schematic illustration of a magnetic resonance imaging system according to an exemplary aspect of the disclosure,

FIG. 7 shows a chart which illustrates a measured deviation between two k-space scanning directions as a function of the number of gradient pulses applied, and

FIG. 8 shows images of a reference recording with artifacts which depend on the number of gradient pulses applied.

### DETAILED DESCRIPTION

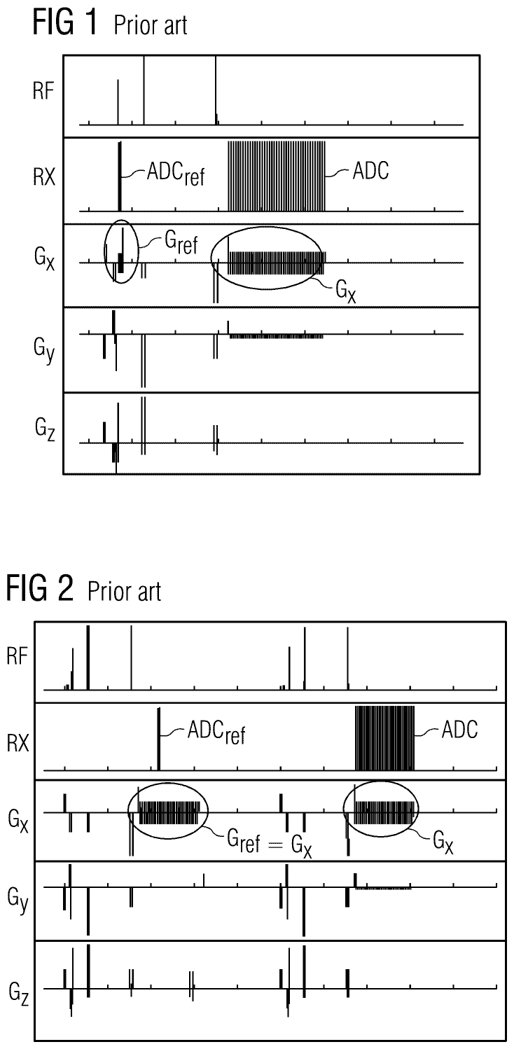

FIG. 1 shows a schematic illustration of a pulse sequence diagram of an echo-planar sequence with a conventional internal phase correction. In the top line, the pulse sequence diagram shows a series of radio-frequency excitation pulses RF. In the second line, labeled as “RX”, the pulse sequence diagram shows a first readout pulse ADC_{ref}, which represents a reference signal, and a series of readout radio-frequency pulses ADC, which represent the magnetic resonance signals of the imaging. In the third line are shown readout gradients Gₓ both for the reference recording G_{ref} (G_{ref} is also referred to as reference gradient in the following) and for the imaging Gₓ (Gₓ here has the meaning of a gradient in readout direction, which is applied simultaneously with the time window for capturing the readout radio-frequency pulse ADC). In the fourth line are illustrated phase-encoding gradients G_{y}. In the fifth line are shown slice selection gradients G_{z}. As mentioned above, the time required for the actual reference measurement is very short, and therefore the reference gradient G_{ref} does not correspond exactly to the gradient Gₓ in readout direction that is applied for the imaging.

FIG. 2 shows a schematic illustration of a pulse sequence diagram of a conventional external phase correction. In the case of external phase correction, a gradient Gₓ in readout direction is already applied during the reference measurement as reference gradient G_{ref}, which corresponds to the gradient Gₓ in readout direction that is applied simultaneously with the readout radio-frequency pulse ADC. In this way, during the reference measurement, interference signals that are produced by the gradient Gₓ in readout direction, which is also applied during the actual imaging, are captured at the same time as the first readout pulse ADC_{ref} that is produced by a radio-frequency reference signal, such that the determination of the interference effects and compensation thereof is improved in comparison with the time-saving method shown in FIG. 1. However, the external phase correction requires more time than the internal phase correction shown in FIG. 1.

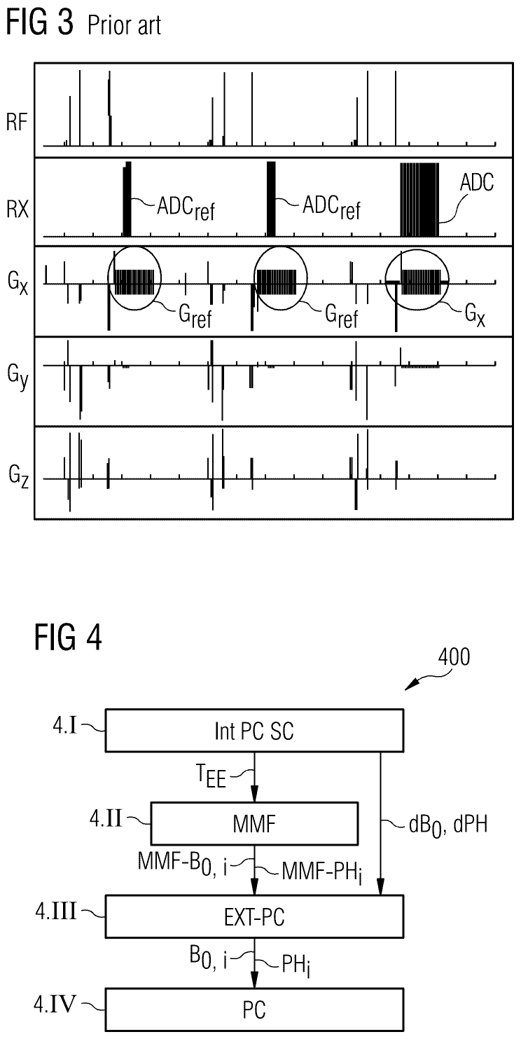

FIG. 3 shows a schematic illustration of a pulse sequence diagram of a conventional phase correction, which is effected on the basis of a GRAPPA reference recording. As part of the conventional correction method shown in FIG. 3, two reference gradients G_{ref} in the readout direction are applied. The reference measurements or the readout pulses ADC_{ref} are used to calculate kernels, which are incorporated during the image reconstruction and are intended to compensate for the interference effects that already occur in the context of the reference measurements.

FIG. 4 shows a flow diagram 400 which illustrates a method for performing magnetic resonance imaging of an examination region using an echo-planar sequence in accordance with an exemplary aspect of the disclosure.

In the step 4.I, a reference recording Int PC SC is initially made, which corresponds to the reference measurement of the “3-line B₀” correction or internal phase correction illustrated in FIG. 1. In addition to a deviation dB₀ of the basic magnetic field and a change dPH of the phase response or a change of the phase, this recording is used to determine a temporal echo-to-echo interval T_{EE}.

In the step 4.II, a mechanical model MMF of the magnetic field generation unit is generated on the basis of the temporal echo-to-echo interval T_{EE}. The mechanical model MMF represents the mechanical oscillatory characteristics of the magnetic field generation unit in the form of a harmonic oscillator. On the basis of this model, it is possible to estimate the contribution MMF-B_{0,i} of the eddy currents resulting from the oscillations to the production of the basic magnetic field B₀ in the form of a model, and it is possible to calculate an estimated phase response or an estimated phase MMF-PH; as a temporal shift in the time domain as a function of a k-space line and the excitation frequency.

On the basis of this model, in the step 4.III an extended correction EXT-PC is performed in respect of the deviation dB₀ of the basic magnetic field already determined in the step 4.I and the change dPH of the phase response determined in the step 4.I, and corrected values for the basic magnetic field B_{0,i} and for the phase response PHᵢ or the phase are thus calculated as a function of the i^{-th} k-space line.

In the step 4.IV, a phase correction PC of the magnetic resonance raw data and hence also of the image data is affected on the basis of the corrected data B_{0,i}, PHᵢ.

FIG. 5 shows a flow diagram that illustrates a method for performing magnetic resonance imaging using an echo-planar sequence in accordance with an alternative exemplary aspect of the disclosure.

In the exemplary aspect illustrated in FIG. 5, the step 5.II additionally includes a one-off use of a phase change scan PC-S, which is complete or recorded in the steady state, and a determination of the k-space line-dependent correction values B₀ᵢ, PHᵢ on the basis of the measured data produced thus. The measured data is obtained from the examination region ROI. The values thus produced are used in the step 5.III as basic values for the mechanical model MMF of the magnetic field generation unit. The steps 5.IV and 5.V correspond to the steps 4.III and 4.IV.

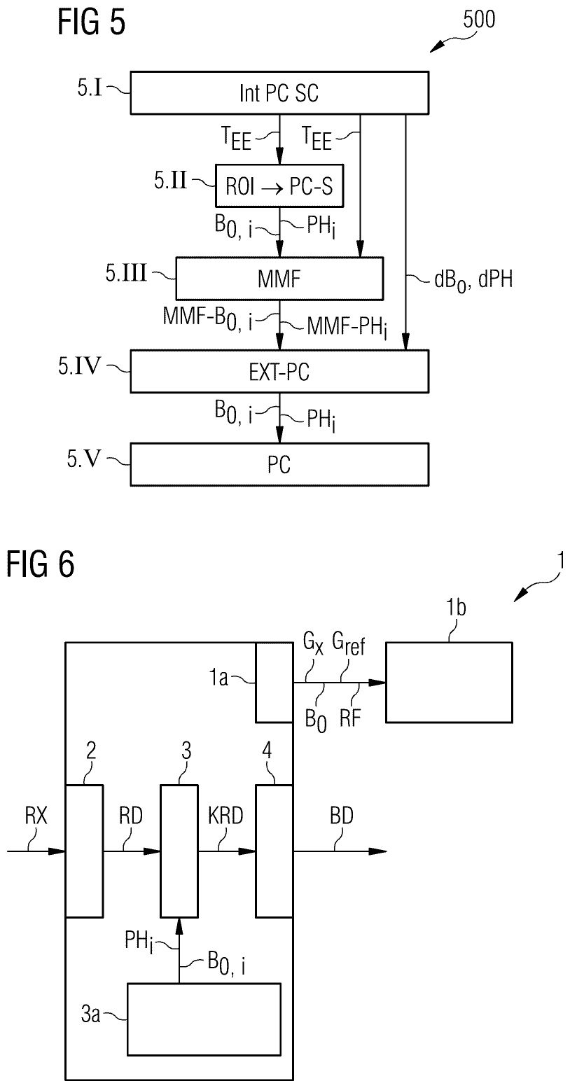

FIG. 6 shows a schematic illustration of a magnetic resonance imaging system 1 according to an exemplary aspect of the disclosure. The magnetic resonance imaging system 1 has a modeling unit 3a, which is configured to generate a theoretical model MMF that represents an oscillating structure of a magnetic field generation unit. In this case, oscillation of the oscillating structure causes eddy currents to be induced in the oscillating structure, said eddy currents provoking a gradient-type interference magnetic field in the examination region ROI. The modeling unit 3a is therefore additionally configured in particular to determine the gradient-type interference magnetic field on the basis of the oscillation of the oscillating structure which has been calculated in the form of a model and the resulting eddy currents of the theoretical model. A change of the basic magnetic field B_{0,i} and of the phase response PHᵢ of the magnetic resonance signals is derived on the basis of the eddy currents or the resulting interference magnetic field.

A further part of the magnetic resonance imaging system 1 is a sequence unit 1a for applying an echo-planar sequence for generating radio-frequency signals RF by means of which magnetic resonance signals RX are excited. The magnetic resonance imaging system 1 also comprises a magnetic field generation unit 1b, which produces a basic magnetic field B₀, gradient fields Gₓ, G_{ref} and radio-frequency fields on the basis of the applied echo-planar sequence.

The magnetic resonance imaging system 1 further comprises an input interface 2 for receiving the magnetic resonance signals RX from the examination region ROI, magnetic resonance raw data RD being generated on the basis of the magnetic resonance signals RX.

A further part of the magnetic resonance imaging system 1 is a correction unit 3 for determining corrected magnetic resonance raw data KRD on the basis of the magnetic resonance raw data RD and on the basis of the deviations determined by the modeling unit 3a in respect of the basic magnetic field B_{0,i} and the phase response PHᵢ of the magnetic resonance signals RX.

The magnetic resonance imaging system 1 also comprises a reconstruction unit 4 for reconstructing magnetic resonance image data BD on the basis of the corrected magnetic resonance raw data KRD.

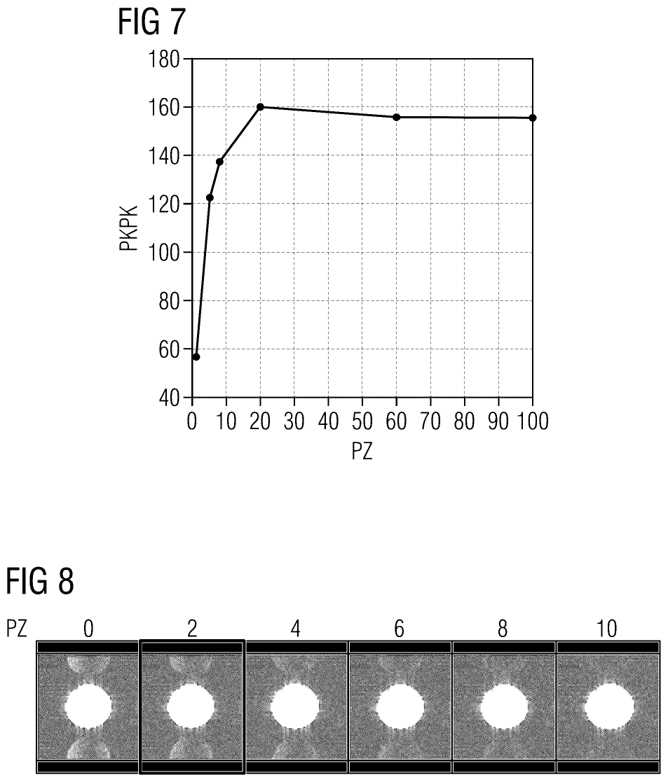

FIG. 7 shows a chart which illustrates a measured deviation PKPK between two k-space scanning directions as a function of the number PZ of bipolar gradient pulses applied. The deviation PKPK relates to the deviation of the basic magnetic field in Hz and from peak to peak. In the scenario illustrated in FIG. 7, a phantom is positioned approximately 15 cm outside the isocenter in x-direction and y-direction. A settling process is completed after a number PZ of 20 bipolar gradient pulses, this being illustrated by the horizontal course of the PKPK curve at the value PZ=20.

FIG. 8 shows images of a reference recording with artifacts which depend on the number of gradient pulses applied. It is evident in FIG. 8 that the artifacts reduce after a number PZ of approximately 10 gradient oscillation periods.

In conclusion, it is again noted that the methods and devices described above are merely preferred exemplary aspects of the disclosure and that the disclosure can be varied by a person skilled in the art without departing from the scope of the disclosure, to the extent that said scope is specified in the claims. For the sake of completeness, it is noted that use of the indefinite article “a” or “an” does not preclude multiple instances of the features concerned. Likewise, the term “unit” does not preclude this consisting of multiple components, which can also be spatially distributed if applicable. Independent of the grammatical term usage, individuals with male, female or other gender identities are included within the term.

The invention claimed is:

1. A method for performing magnetic resonance imaging of an examination region using an echo-planar sequence, comprising:
generating a theoretical model which represents an oscillating structure of a magnetic field generation unit based on the echo-planar sequence, wherein as a result of an oscillation of the oscillating structure, eddy currents which produce a gradient-type interference magnetic field in the examination region are induced in the oscillating structure;
determining the gradient-type interference magnetic field based on the theoretical model;
applying the echo-planar sequence;
receiving magnetic resonance signals from the examination region, wherein magnetic resonance raw data is generated based on the magnetic resonance signals;
determining corrected magnetic resonance raw data based on the generated magnetic resonance raw data and based on the determined gradient-type interference magnetic field in the examination region; and
reconstructing magnetic resonance image data based on the corrected magnetic resonance raw data.

2. The method as claimed in claim 1, wherein the theoretical model comprises a mechanical model that describes mechanical oscillatory characteristics of the magnetic field generation unit with which the magnetic resonance imaging is performed.

3. The method as claimed in claim 1, wherein the determination of the corrected magnetic resonance raw data comprises a phase correction.

4. The method as claimed in claim 1, wherein the eddy currents comprise fast-decaying eddy currents having a time constant between 0.1 and 10 ms.

5. The method as claimed in claim 1, wherein the theoretical model of the oscillating structure comprises an attenuated harmonic oscillator.

6. The method as claimed in claim 1, wherein the echo-planar sequence comprises a reference measurement for a phase change.

7. The method as claimed in claim 6, wherein the reference measurement for the phase change comprises a complete reference recording of k-space.

8. The method as claimed in claim 6, wherein the reference measurement for the phase change takes place in steady state.

9. The method as claimed in claim 1, wherein the echo-planar sequence comprises an initial reference measurement for identifying drift effects.

10. The method as claimed in claim 1, wherein the echo-planar sequence comprises a reference measurement for a phase change, a temporal echo-to-echo interval is determined based on the reference measurement, and the theoretical model is determined based on the temporal echo-to-echo interval.

11. The method as claimed in claim 1, wherein the echo-planar sequence comprises a reference measurement for a phase change, which includes a scan of at least three reference lines.

12. A magnetic resonance imaging system, comprising:
a modeling unit configured to generate a theoretical model which represents an oscillating structure of a magnetic field generation unit based on an echo-planar sequence that is to be applied, wherein as a result of an oscillation of the oscillating structure, eddy currents are induced in the oscillating structure and produce a gradient-type interference magnetic field in an examination region, and to determine the gradient-type interference magnetic field based on the theoretical model;
a sequence unit configured to apply the echo-planar sequence;
an input interface configured to receive magnetic resonance signals from the examination region, wherein magnetic resonance raw data is generated based on the magnetic resonance signals;
a correction unit configured to determine corrected magnetic resonance raw data based on the magnetic resonance raw data and based on the determined gradient-type interference magnetic field in the examination region; and
a reconstruction unit configured to reconstruct magnetic resonance image data based on the corrected magnetic resonance raw data.

13. A non-transitory computer-readable storage medium comprising instructions which, when executed by a computer, cause the computer to execute the steps of the method as claimed in claim 1.
